(19) United States
(12) Patent Application Publication
(10) Pub. No.: US 2026/0295180 A1
(43) Pub. Date: Oct. 1, 2026

(54) SYSTEM AND METHOD FOR PREDICTING INSUFFLATOR BOTTLE GAS TIME REMAINING

(71) Applicant: Conmed Corporation, Largo, FL (US)

(72) Inventors: Michael L. Koltz, Aurora, CO (US); Sami Al-Abed, Glendale, CO (US)

(73) Assignee: Conmed Corporation, Largo, FL (US)

(21) Appl. No.: 18/996,473

(22) PCT Filed: Jul. 13, 2023
(86) PCT No.: PCT/US2023/027658

§ 371 Date: Jan. 17, 2025

Related U.S. Application Data
(60) Provisional application No. 63/390,078, filed on Jul. 18, 2022.

(51) Int. Cl. A61M 13/00

(52) U.S. Cl. CPC A61M 13/003; A61M 2205/18; A61M 2205/3331; A61M 2205/502

(57) ABSTRACT

A surgical gas delivery system is disclosed that includes an inlet flow path for receiving insufflation gas from a gas bottle, a pressure sensor communicating with the inlet flow path for measuring bottle gas pressure periodically, and a processor communicating with the pressure transducer and configured to monitor a rate of change of the bottle gas pressure to calculate a predicted time before the bottle gas pressure reaches a minimum functional pressure value.

### CROSS-REFERENCE TO RELATED APPLICATIONS

The subject application claims the benefit of priority to U.S. Provisional Patent Application No. 63/390,078 filed Jul. 18, 2022, the disclosure of which is incorporated herein by reference in its entirety.

### BACKGROUND OF THE DISCLOSURE

### 1. Field of the Disclosure

The subject disclosure is directed to endoscopic surgery, and more particularly, to a surgical gas delivery system that is adapted and configured to predict when insufflator bottle gas pressure reaches a minimum functional pressure value.

### 2. Description of Related Art

Laparoscopic or “minimally invasive” surgical techniques are becoming commonplace in the performance of procedures such as cholecystectomies, appendectomies, hernia repair and nephrectomies. Such procedures commonly involve filling or “insufflating” the abdominal cavity with a pressurized fluid, such as carbon dioxide, to create an operating space, which is referred to as a pneumoperitoneum.

Insufflators are used to deliver insufflation gas, typically CO₂ or another medical gas, from a gas source to a surgical cavity. Oftentimes, the gas source is a gas bottle or a portable tank. Insufflators typically quantify and display the total volume of insufflation gas dispensed or delivered to the surgical cavity from a gas bottle. This total volume can be used by an experienced user to infer or otherwise estimate the amount of time remaining before the gas bottle is empty and/or must be replaced.

Gas bottle switching or exchange must be done by personnel, such as circulators, outside of the sterile field. If the gas bottle empties when a circulator is unavailable, a delay in surgery occurs and pneumoperitoneum must be re-established to continue surgery. The system and method described herein repeatedly calculates and displays the time remaining until the gas bottle must be exchanged, removing the task of estimating or calculating from operating room personnel.

### SUMMARY OF THE DISCLOSURE

The subject disclosure is directed to a new and useful surgical gas delivery system that is adapted and configured to determine or otherwise predict an amount of insufflator bottle gas time remaining for a surgical procedure, removing that burden from operating room personnel.

The gas delivery system includes an inlet flow path for receiving insufflation gas from a gas bottle, a pressure sensor communicating with the inlet flow path for measuring bottle gas pressure periodically, and a processor communicating with the pressure transducer and configured to monitor a rate of change of the bottle gas pressure to calculate a predicted time before the bottle gas pressure reaches a minimum functional pressure value.

The system further includes a temperature sensor communicating with the inlet flow path to measure bottle gas temperature periodically. These periodic gas temperature measurements can be used by the processor to improve prediction accuracy. The system also includes a visual display or graphical user interface for indicating an amount of time until the bottle gas pressure reaches the minimum functional pressure value. A sound transducer communicates with the processor for producing an audible alarm signal when the bottle gas pressure reaches the minimum functional pressure value.

In an embodiment of the disclosure, the processor uses a mathematical function to calculate the predicted time before the bottle gas pressure reaches the minimum functional pressure value. The mathematical function can be a linear regression function, a non-linear regression function or a curve fitting function. In another embodiment of the disclosure, the processor uses a look up table to estimate an equivalent gas volume based on a measured gas bottle pressure or gas bottle temperature.

The subject disclosure is also directed to a novel computer-implemented method for predicting when the insufflator bottle gas pressure delivered to a surgical gas delivery device will reach a minimum functional pressure value, which includes the steps of receiving insufflation gas from a gas bottle through an inlet flow path of the surgical gas delivery device, periodically measuring bottle gas pressure through the inlet flow path, monitoring a rate of change of the bottle gas pressure to calculate a predicted time before the bottle gas pressure reaches a minimum functional pressure value, and indicating an amount of time until the bottle gas pressure reaches the minimum functional pressure value, which can be a visible or audible indication.

These and other features of the systems and methods of the subject disclosure will become more readily apparent from the following detailed description of the disclosed embodiments taken in conjunction with the drawings.

### BRIEF DESCRIPTION OF THE DRAWINGS

So that those skilled in the art will readily understand how to make and use the gas delivery system of the subject disclosure without undue experimentation, preferred embodiments thereof will be described in detail herein below with reference to the figures wherein:

FIG. 1 is an illustration of a surgical gas delivery device or insufflator having a graphical user interface screen or visual display configured to indicate a time remaining before the bottle gas pressure reaches a minimum functional pressure value and the gas bottle must be exchanged or otherwise replaced;

FIG. 1A is an illustration of the graphical user interface screen shown in FIG. 1, depicting a symbolic element in the form of a dynamically changing graphic to indicate an amount of time until the bottle gas pressure reaches the minimum functional pressure value;

FIG. 2 is a schematic representation of the surgical gas delivery system of the subject disclosure, which is adapted and configured to predict when bottle gas pressure reaches a minimum functional pressure value;

FIG. 3 is a curve plot of pressure response versus time using a linear regression technique; and

FIG. 4 is a curve plot of pressure response versus time using a non-linear regression technique.

### DETAILED DESCRIPTION OF THE EMBODIMENTS

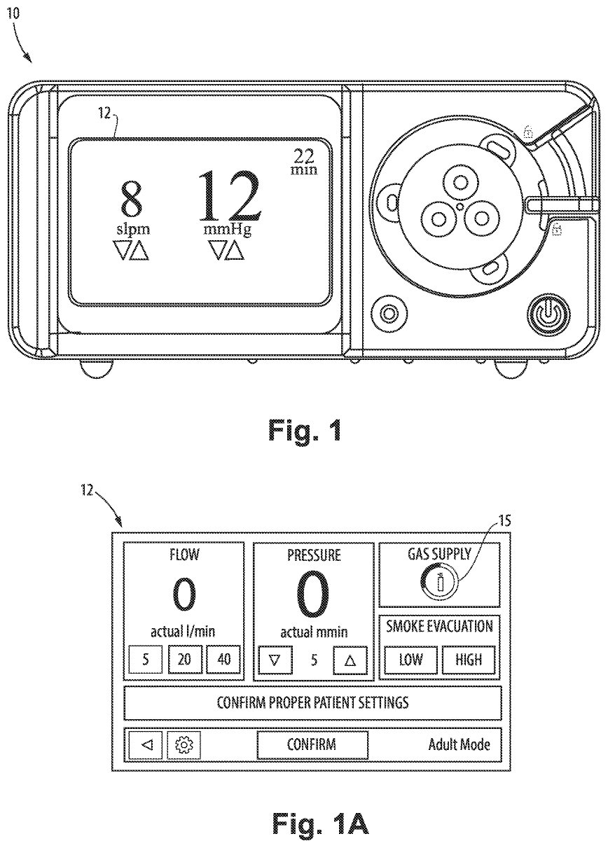

Referring now to the drawings wherein like reference numerals identify similar features of the devices and systems disclosed herein, there is illustrated in FIG. 1 a surgical gas delivery device or insufflator 10 for delivering insufflation gas from a gas source, such as a gas bottle or tank, to a body cavity of a patient during a surgical procedure. An example of a gas delivery device of this type is disclosed in commonly assigned U.S. Pat. No. 9,199,047, the disclosure of which is incorporated herein by reference in its entirety.

Gas delivery device 10 has a graphical user interface screen or visual display 12 that is adapted and configured to indicate a time remaining before the bottle gas pressure in a gas bottle reaches a minimum functional pressure value. In this exemplary illustration, there is 22 minutes remaining until the bottle gas pressure reaches the minimum functional pressure. At such a time the gas bottle or tank would need to be exchanged or otherwise replaced.

FIG. 1A illustrates the graphical user interface screen 12 of gas delivery device 10 shown in FIG. 1, displaying a symbolic element in the form of a dynamically changing annular graphic element 15. The annular graphic element 15 symbolically indicates an amount of time until the bottle gas pressure reaches the minimum functional pressure value. Other symbolic or graphical elements to indicate an amount time may be used.

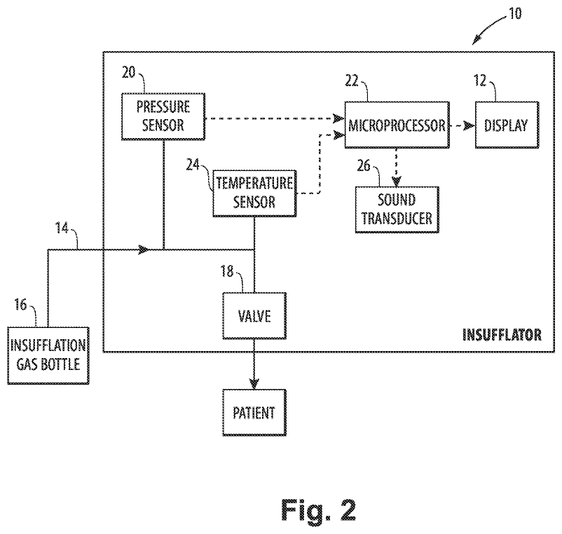

Referring now to FIG. 2, there is illustrated a schematic representation of the gas delivery device 10. It includes an inlet flow path 14 for receiving insufflation gas from a gas bottle 16 and for delivering that gas to a valve 18 that controls the flow of gas to the body cavity of a patient. The valve 18 can be a solenoid valve having on/off flow control or a proportional valve configured to dynamically control the delivery of insufflation gas to the patient's body cavity.

A pressure sensor 20 communicates with the inlet flow path 14 for measuring bottle gas pressure. Pressure can be measured continuously or periodically at a predefined polling frequency. It is envisioned that the processor 22 would read the pressure signal at a polling frequency of 1 kHz to determine the rate of change of the bottle gas pressure. However, the polling frequency could be between 1 GHz and 0.01 Hz.

A programmable processor 22 communicates with the pressure sensor 20 and it is configured to monitor a rate of change of the bottle gas pressure. The rate of change of bottle gas pressure is then used by the processor 22 to calculate a predicted time before the bottle gas pressure reaches a minimum functional pressure value, as will be discussed in greater detail below.

With continuing reference to FIG. 2, the surgical gas delivery system 10 also includes a temperature sensor 24 that communicates with the inlet flow path 14 to measure bottle gas temperature. This too can be done continuously or periodically. The temperature measurements are used by the processor 22 to improve prediction accuracy.

The gas delivery system 10 further includes a sound transducer 26 that communicates with the processor 22 for producing an audible alarm signal when the bottle gas pressure reaches the minimum functional pressure value. The sound transducer 26 will alert the user of a low time remaining condition. Additionally, the operator can set the processor 22 to produce an audible alert when the calculated gas time remaining drops below a specific level. It is envisioned that a visual alarm could also be provided. For example, an indicator light could be provided on the display screen 12.

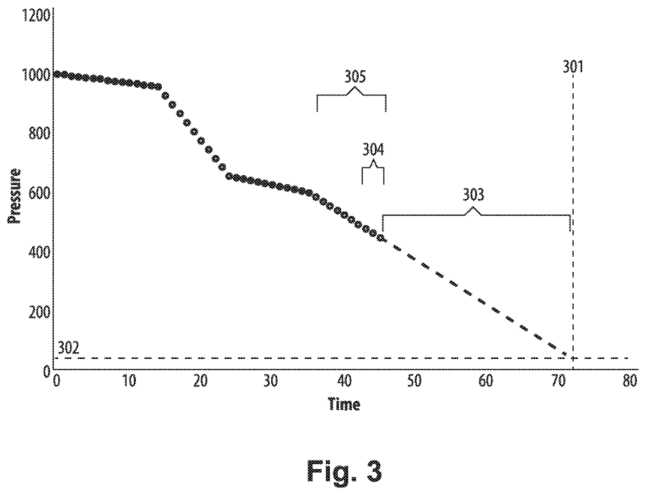

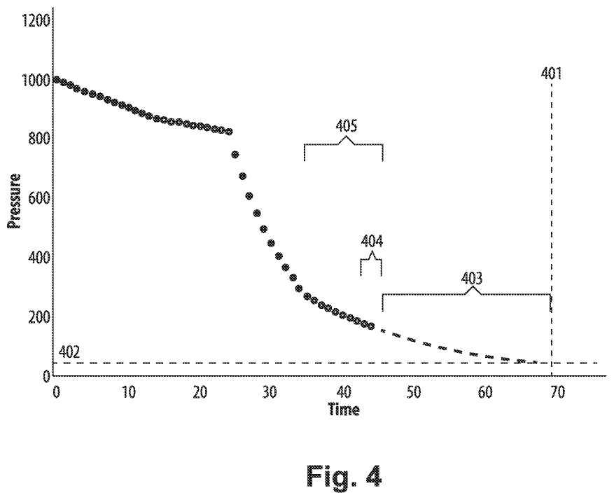

In accordance with the subject disclosure, the processor 22 uses a mathematical function to calculate the predicted time before the bottle gas pressure reaches the minimum functional pressure value. More particularly, the processor 22 performs computer-implemented calculations with respect to time to predict the amount of time until the bottle pressure reaches the minimum functional pressure. In this regard, the processor 22 can employ a linear regression technique as shown for example in FIG. 3 or a non-linear regression technique as shown for example in FIG. 4.

Alternatively, the processor 22 could employ a look-up table stored in memory to estimate a standard equivalent volume based on a measured gas bottle pressure and/or a measured gas bottle temperature. Here, the temperature measurements will improve the predictive accuracy of the system

In the simplest embodiment, the insufflation gas is assumed to be an ideal gas in which a unit decrease in pressure corresponds to constant decrease in gas volume. Some insufflation gases, such as carbon dioxide, exhibit far from ideal gas changes in volume with respect to pressure and temperature. To account for this non-linear relationship, a non-linear regression or curve fitting function can be used.

Referring to FIGS. 3 and 4, the time until the bottle gas reaches a functional minimum pressure (e.g., 50 psi) is calculated by a linear regression technique or a non-linear regression technique 303, 403 that use an input of multiple recent pressure measurements 304, 404 (and temperature measurements) to estimate the time 301, 401 at which the corresponding curve or function will reach a minimum system functional pressure 302, 402.

The gas delivery device 10 displays the calculated time until the functional minimum pressure is achieved and updates the displayed value periodically as new pressure and temperature data is received. As new data is received, the oldest data is removed from the calculation.

During insufflation, there are generally periods of approximately constant leak rate compensation 305, 405, with the periodic leak rate varying from period to period. This behavior lends itself to using regression techniques to calculate the time remaining until the functional minimum bottle gas pressure is reached. Although the calculated time can vary greatly as the periodic leak rate changes, the absolute time error approaches zero as conditions approach the functional minimum.

The disclosed gas delivery system offers an advantage over the prior art in that it does not require an operator to estimate when the gas bottle contents will be consumed. This requires experience and repeated observation of the insufflator display in addition to surgical conditions influencing leak rate. Elimination of operator experience from the effort of estimating gas bottle exhaustion provides for the use of new techniques, procedures, and personnel without degrading the accuracy of gas depletion estimation.

As will be appreciated by those skilled in the art, aspects of the present disclosure may be embodied as a system, method, or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object-oriented programming language such as Java, Smalltalk, C++, or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus, or other devices to produce a computer implemented process such that the instructions which execute on the computer or other programmable apparatus provide processes for implementing the functions/acts specified herein.

While the subject disclosure has been shown and described with reference to preferred embodiments, those skilled in the art will readily appreciate that changes and/or modifications may be made thereto without departing from the spirit or scope of the subject disclosure.

What is claimed is:

1. A surgical gas delivery system comprising:
a) an inlet flow path for receiving insufflation gas from a gas bottle;
b) a pressure sensor communicating with the inlet flow path for measuring bottle gas pressure periodically; and
c) a processor communicating with the pressure transducer and configured to monitor a rate of change of the bottle gas pressure to calculate a predicted time before the bottle gas pressure reaches a minimum functional pressure value.

2. A surgical gas delivery system as recited in claim 1, wherein the pressure sensor measures bottle gas pressure at a polling frequency of 1 kHz to determine the rate of change of the bottle gas pressure.

3. A surgical gas delivery system as recited in claim 1, wherein the pressure sensor measures bottle gas pressure at a polling frequency of between 1 GHZ and 0.01 Hz to determine the rate of change of the bottle gas pressure.

4. A surgical gas delivery system as recited in claim 1, further comprising a temperature sensor communicating with the inlet flow path to measure bottle gas temperature periodically, wherein the processor monitors bottles gas temperature to improve prediction accuracy.

5. A surgical gas delivery system as recited in claim 1, further comprising a visual display communicating with the processor for indicating an amount of time until the bottle gas pressure reaches the minimum functional pressure value.

6. A surgical gas delivery system as recited in claim 1, wherein the visual display provides a numeric value to indicate an amount of time until the bottle gas pressure reaches the minimum functional pressure value.

7. A surgical gas delivery system as recited in claim 1, wherein the visual display provides a symbolic element to indicate an amount of time until the bottle gas pressure reaches the minimum functional pressure value.

8. A surgical gas delivery system as recited in claim 7, wherein the symbolic element is a dynamically changing graphic to indicate an amount of time until the bottle gas pressure reaches the minimum functional pressure value.

9. A surgical gas delivery system as recited in claim 1, further comprising a sound transducer communicating with the processor for producing an audible alarm signal when the bottle gas pressure reaches the minimum functional pressure value.

10. A surgical gas delivery system as recited in claim 1, further comprising a valve for controlling an outflow of insufflation gas to a patient.

11. A surgical gas delivery system as recited in claim 1, wherein the processor uses a mathematical function to calculate the predicted time before the bottle gas pressure reaches the minimum functional pressure value.

12. A surgical gas delivery system as recited in claim 11, wherein the mathematical function is a linear regression function.

13. A surgical gas delivery system as recited in claim 11, wherein the mathematical function is a non-linear regression function.

14. A surgical gas delivery system as recited in claim 11, wherein the mathematical function is a curve fitting function.

15. A surgical gas delivery system as recited in claim 1, wherein the processor uses a look-up table to estimate an equivalent volume based on a measured gas bottle pressure or gas bottle temperature.

16. A computer-implemented method for predicting when the bottle gas pressure delivered to a surgical gas delivery device will reach a minimum functional pressure value comprising:
a) receiving insufflation gas from a gas bottle through an inlet flow path of the surgical gas delivery device;
b) periodically measuring bottle gas pressure through the inlet flow path;
c) monitoring a rate of change of the bottle gas pressure to calculate a predicted time before the bottle gas pressure reaches a minimum functional pressure value; and
d) indicating an amount of time until the bottle gas pressure reaches the minimum functional pressure value.

17. A method according to claim 16, wherein a mathematical function is used to calculate the predicted time before the bottle gas pressure reaches the minimum functional pressure value, and wherein the mathematical function is a linear regression function.

18. A method according to claim 16, wherein a mathematical function is used to calculate the predicted time before the bottle gas pressure reaches the minimum functional pressure value, and wherein the mathematical function is a non-linear regression function.

19. A method according to claim 16, wherein a mathematical function is used to calculate the predicted time before the bottle gas pressure reaches the minimum functional pressure value, and wherein the mathematical function is a curve fitting function.

20. A method according to claim 16, wherein a look-up table is used to estimate an equivalent volume based on a measured gas bottle pressure or gas bottle temperature.
